(19) United States
(12) Patent Application Publication
(10) Pub. No.: US 2011/0280877 A1
(43) Pub. Date: Nov. 17, 2011

(54) Inhibition of B7-H1/CD80 interaction and uses thereof

(76) Inventor: Koji Tamada, Lutherville, MD (US)

(21) Appl. No.: 13/068,337

(22) Filed: May 10, 2011

Related U.S. Application Data
(60) Provisional application No. 61/333,294, filed on May 11, 2010.

(51) Int. Cl. A61K 39/395; A61P 31/18; A61P 35/00; A61P 31/12; C07K 16/18; A61K 39/00

(52) U.S. Cl. 424/134.1; 530/388.1; 424/141.1; 424/277.1; 424/133.1

(57) ABSTRACT

The present invention provides a composition comprising an agent which specifically blocks interaction between B7-H1 and CD80 but not interaction between B7-H1 and PD-1 and a vaccine, optionally in a pharmaceutically acceptable carrier. Further provided is a method of treating or inhibiting abnormal cell proliferation or a viral infection in a host comprising the step of administering an agent which specifically blocks interaction between B7-H1 and CD80 but does not block interaction between B7-H1 and PD-1 in combination with a vaccine against the cancer to a host in need thereof.

### CROSS-REFERENCE TO RELATED APPLICATION

This nonprovisional application claims benefit of priority under 35 U.S.C. §119(e) of provisional applications U.S. Ser. No. 61/333,294, filed May 11, 2010, now abandoned, the entirety of which is hereby incorporated by reference.

### FEDERAL FUNDING LEGEND

This invention was made with government support under Grant Number HL088954 awarded by the National Institutes of Health. The government has certain rights in the invention.

### BACKGROUND OF THE INVENTION

1. Field of the Invention

The present invention relates to the fields of T cell physiology and cancer. More specifically, the present invention relates to, inter alia, inhibition of B7-H1/CD80 interaction and uses thereof.

2. Description of the Related Art

B7-H1 (CD274, PD-L1), a transmembrane glycoprotein belonging to Ig superfamily molecule, plays an integral role in the regulation of immune tolerance and homeostasis (1). Mice deficient of B7-H1 gene or wild-type mice treated with anti-B7-H1 blocking mAb exhibited exacerbated autoimmune phenotypes associated with an activation of self-reactive CD4⁴⁺ and CD8⁺ T cells (2-5). Tolerogenic functions of B7-H1 are dependent on its expression on hematopoietic or parenchymal cells, and mediated by its interaction with PD-1 receptor (6-8). PD-1 is inducibly expressed on T cells after activation and delivers co-inhibitory signals via immunoreceptor tyrosine-based switch motif in the cytoplasmic domain (9-10). PD-1 signal interferes with phosphatidylinositol-3-kinase (Pl3K) activity and subsequently inhibits IL-2 production, which eventually renders T cells anergic (11). The mice deficient of PD-1 gene spontaneously develop autoimmune phenotypes, and single nucleotide polymorphisms of human PD-1 gene are associated with an increased risk of autoimmune diseases (12-16).

Recent studies by Butte et al. discovered that B7-H1 interacts with CD80 (B7-1) in addition to PD-1 (17-18). In vitro studies using CD4+T cells deficient of PD-1, CD28, and/or CTLA-4 indicated that B7-H1/CD80 interaction delivers bidirectional inhibitory signals to T cells (17). These findings are consistent with previous observations implicating the presence of non-PD-1 receptor(s) of B7-H1. For instance, when the B7-H1/PD-1 interaction is blocked in models of T cell tolerance, the effects of anti-B7-H1 antagonistic mAb in restoring T cell functions were more vigorous than that mediated by anti-PD-1 antagonistic mAb (19-20). These results have been observed in multiple experimental systems using distinct clones of anti-B7-H1 and PD-1 mAbs. However, it remains unknown whether CD80 interaction with B7-H1 is responsible for these observations and, if so, how this interaction affects T cell tolerance in physiological or pathological conditions in vivo.

Potential difficulties of functional studies of the B7-H1/CD80 pathway reside in its complexity of the ligand-receptor interactions. B7-H1 binds both PD-1 and CD80, while CD80 interacts with CD28 and CTLA-4 in addition to B7-H1. Thus, genetic ablation of B7-H1 or CD80 results in a loss of multiple receptor interactions and hardly addresses selective functions of B7-H1/CD80 pathway.

Thus, there is a lack in the prior art of methods and therapies that specifically interfere with the B7-H1/CD80 interaction but not the B7-H1/PD-1 interaction. The present invention fulfills this long-standing need and desire in the art.

### SUMMARY OF THE INVENTION

The present invention teaches that attenuation of B7-H1/CD80 signals by treatment with anti-B7-H1 monoclonal antibody, which specifically blocks B7-H1/CD80 but not B7-H1/PD-1, enhanced T cell expansion and prevented T cell anergy induction. In addition, B7-H1/CD80 blockade restored Ag responsiveness in the previously anergized T cells. Experiments using B7-H1 or CD80-deficient T cells indicated that an inhibitory signal through CD80, but not B7-H1, on T cells is responsible in part for these effects.

Consistently, CD80 expression was detected on anergic T cells and further upregulated when they were re-exposed to the Ag. Finally, blockade of B7-H1/CD80 interaction prevented oral tolerance induction and restored T cell responsiveness to Ag previously tolerized by oral administration. Taken together, the present invention demonstrates that the B7-H1/CD80 pathway is a crucial regulator in the induction and maintenance of T cell tolerance.

Thus, the present invention is directed to an anti-B7-H1 monoclonal antibody which specifically blocks interaction between B7-H1 and CD80 but not interaction between B7-H1 and PD-1.

In another embodiment, the present invention provides a method of enhancing T cell expansion and decreasing T cell anergy induction in an individual in need of such treatment, comprising the step of administering to an individual an effective amount of a monoclonal antibody which specifically blocks interaction between B7-H1 and CD80 but not interaction between B7-H1 and PD-1.

In yet another embodiment, the present invention provides a method of enhancing efficacy of a vaccine comprising administering an agent which specifically blocks interaction between B7-H1 and CD80 but not interaction between B7-H1 and PD-1.

In yet another embodiment, the present invention provides a method of treating or inhibiting abnormal cell proliferation or a viral infection in a host comprising the step of administering an agent which specifically blocks interaction between B7-H1 and CD80 but not interaction between B7-H1 and PD-1 in combination with a vaccine against the cancer to a host in need thereof.

In still yet another embodiment, the present invention provides a composition comprising an agent which specifically blocks interaction between B7-H1 and CD80 but not interaction between B7-H1 and PD-1 and a vaccine, optionally in a pharmaceutically acceptable carrier.

Other and further aspects, features and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIGS. 1A-1E show selective blockade of B7-H1/CD80 interaction by anti-B7-H1 mAb clone 43H12. FIG. 1A shows 293T cells transfected with mock or mouse B7-H1-encoding plasmids were stained with 1 μg/ml anti-B7-H1 mAb clone 43H12 (black histogram) or control rat IgG (gray histogram) followed by FITC-conjugated anti-rat IgG. Binding of 43H12 to B7-H1 was analyzed by flow cytometry. FIG. 1B shows an ELISA plate was coated with 2 μg/ml mouse B7-H1-Fc (closed circle), mouse CD8O-Fc (open circle), mouse B7-DC-Fc (open square), mouse B7-H3-Fc (closed triangle), or mouse 87-H4-Fc (closed diamond) fusion proteins. Indicated doses of 43H12 were added into wells and its binding with the coated proteins were detected by HRP-conjugated anti-rat IgG Ab. Average +/− SD of O.D. from triplicate wells are shown. FIG. 1C shows 293T cells transfected with plasmids encoding mock (gray histogram) or B7-H1 (black histogram) were incubated with 2 μg/ml biotin-conjugated CD8O-Fc (left panels) or PD-1-Fc (right panels) fusion proteins in the presence of 2 μg/ml 43H12, 10B5, or control rat IgG. The staining of fusion proteins were detected by streptavidin-PE in flow cytometry. FIG. 1D shows 293T cells transfected with plasmids encoding B7-H1 were stained with CD8O-Fc (open circle) or PD-1-Fc (closed circle) fusion proteins in the presence of indicated doses of 43H12. Percentage of positively stained cells was assessed by flow cytometry. FIG. 1E shows T cells isolated from CD80-KO mice were stimulated with anti-CD3 mAb together with immobilized B7-H1-Fc (filled column) or control human Fc (open column) in the presence of soluble 43H12 or control rat IgG. Proliferation of the culture cells were assessed by ³H-thymidine incorporation. All experiments were repeated at least 3 times and a representative data is shown.

FIGS. 2A-2C show enhanced expansion of Ag-reactive CD8⁺ T cells by blockade of B7-H1/CD80 interaction. B6 mice were transferred i.v. with OT-I T cells and injected i.v. with 0.5 mg OVA₂₅₇₋₂₆₄ peptide. On the day of peptide injection and 3 days later, the mice were treated i.p. with 200 μg 43H12 or control rat IgG. FIG. 2A: PBMC was harvested at the indicated time points, and a percentage of OT-I T cells in total CD8-positive cells was assessed in the 43H12-treated (closed circle) or control IgG-treated (open circle) mice by flow cytometry. The data are shown as mean +/− SEM. FIG. 2B: Mice were given i.p. with 100 μg BrdU on day 2 (upper panels) or day 4 (lower panels) after OVA peptide injection. Twenty-four hrs after BrdU injection, spleen cells were harvested and BrdU incorporation in CD8/OVA-tetramer double-positive OT-I T cells was analyzed by flow cytometry (black histogram). As background level, OT-I T cells in the mice without BrdU administration were stained similarly (gray histogram). FIG. 2C: Spleen cells were harvested 4 days after OVA peptide injection and Annexin V staining in CD8/OVA-tetramer double-positive OT-I T cells was analyzed by flow cytometry (black histogram). Background level without Annexin V staining is also shown (gray histogram). All experiments were independently repeated for at least 3 times, and the representative data are shown. The numbers in the histogram indicate the percentage of positively stained cells.

FIGS. 3A-3C shows role of T cell-associated CD80 in the inhibitory effects of 87-H1/CD80 interaction. FIG. 3A: WT B6 mice or CD80-KO mice were transferred i.v. with OT-I T cells. In FIG. 3B, B6 mice were transferred i.v. with WT, B7-H1-KO, or CD80-KO background OT-I T cells. In both settings, the recipient mice were injected i.v. with 0.5 mg OVA₂₆₇-₂₆₄ peptide, and treated i.p. with 200 μg 43H12 or control rat IgG on day of peptide injection and 3 days later. Splenocytes were harvested 5 days after peptide injection, and the percentage of OT-I T cells in CD8-positive population was assessed by flow cytometry. FIG. 3C: B6 mice were transferred i.v. with OT-I T cells and injected i.v. with 0.5 mg OVA₂₅₇-₂₆₄ peptide. On day 3 and 5, CD8/OVA-tetramer double-positive OT-I T cells was stained with anti-CD80 mAb and analyzed by flow cytometry (grey histogram). Non-stained background levels of the same cells are also shown (open histogram). All experiments were repeated at least 3 times, and the representative data are shown. The numbers in the histogram indicate the percentage of positively stained cells.

FIGS. 4A-4C shows prevention and restoration of CD8⁺ T cell anergy by blockade of B7-H1/CD80 interaction. B6 mice were transferred i.v. with OT-I T cells and injected i.v. with 0.5 mg OVA₂₅₇₋₂₆₄ peptide. FIG. 4A: On day of peptide injection and 3 days later, the mice were treated i.p. with 200 μg 43H12 (filled circle) or control rat IgG (open circle). Thirty-four days after initial peptide injection, the mice were re-challenged i.v. with 0.5 mg OVA₂₅₇₋₂₆₄ peptide, and percentages of CD8/OVA-tetramer double-positive OT-I T cells in PBMC were assessed by flow cytometry at the indicated time points. Fold expansion of OT-I T cells was calculated by dividing OT-I T cell percentages after re-challenge by that before re-challenge in individual mice. FIG. 4A: Twenty days after the initial OVA peptide injection, the mice were re-challenged with 0.5 mg OVA₂₅₇₋₂₆₄ peptide and treated i.p. with 200 μg 43H12 (filled circle) or control rat IgG (open circle) on day of peptide re-challenge and 3 days later. Fold expansion of OT-I T cells in PBMC was assessed as FIG. 4A at the indicated time points. FIG. 4C: Twenty days after the initial OVA peptide injection, the mice were left untreated (left panel) or re-challenged with 0.5 mg OVA₂₅₇₋₂₆₄ peptide (right panel). Twenty four hrs later, CD8/OVA-tetramer double-positive OT-I T cells in the spleen was stained with anti-CD80 mAb and analyzed by flow cytometry (gray histogram). Non-stained background levels of the same cells are also shown (open histogram). All experiments were repeated for at least 3 times and the representative data are shown. The numbers in the histogram indicate the percentage of positively stained cells.

FIGS. 5A-5G show prevention and restoration of oral tolerance by blockade of B7-H1/CD80 interaction. B6 mice were given drinking water supplemented with OVA protein (open or closed circles) or without OVA (open square) from day 0 to 7. On day 14, the mice were immunized s.c. with OVA protein emulsified in CFA. The mice were also treated i.p. with 150 mg 43H12 (closed circle) or control rat IgG (open circle) on day 0, 4, 8, and 12 (FIGS. 5A-5D, 5F:) or on day 14 and 17 (FIG. 5E, 5G). On day 21, draining LN cells were harvested from the mice and cultured with the indicated doses of OVA protein. After 48 hrs, production of IFN-γ (FIGS. 5A, 5E), IL-2 (FIG. 5B) and IL-4 (FIG. 5C) in culture supernatant was measured by ELISA. IL-17 (FIG. 5D) level was measured 24, 48, and 72 hrs after culture with 25 mg/ml OVA protein. Proliferative activity was assessed by an incorporation of ³H-thymidine (FIGS. 5F-5G). All experiments were repeated for at least 3 times. Representative data are shown as mean +/− SD of triplicate wells in each group.

### DETAILED DESCRIPTION OF THE INVENTION

The present invention is directed to an anti-B7-H1 monoclonal antibody which specifically blocks interaction between B7-H1 and CD80 but does not block the interaction between B7-H1 and PD-1. Although desirable effects could be obtained with much less specificity, in a particularly preferred embodiment, the antibody blocks B7-H1/CD80 interaction with at least 30-fold higher specificity than B7-H1/PD-1. A representative example of an anti-B7-H1 monoclonal antibody is 43H12.

The present invention is further directed to a method of enhancing T cell expansion and decreasing T cell anergy induction in an individual in need of such treatment, comprising the step of administering to the individual an effective amount of a monoclonal antibody which specifically blocks interaction between B7-H1 and CD80 but not interaction between B7-H1 and PD-1. Generally, administration of this antibody results in certain desirable biological effects, including but not limited to an enhanced T cell response, decreases the inhibitory effect on late expansion phase of Ag-induced T cell responses and decreases T cell anergy induction and increases production of IL-4 and IL-17.

The present invention is further directed to a method of enhancing the efficacy of a vaccine comprising administering an agent which specifically blocks interaction between B7-H1 and CD80 but not interaction between B7-H1 and PD-1.

The present invention is further directed to a method of treating or inhibiting abnormal cell proliferation or a viral infection in a host comprising the step of: administering an agent which specifically blocks the interaction between B7-H1 and CD80 but does not block the interaction between B7-H1 and PD-1 in combination with a vaccine against the cancer to a host in need thereof. In one embodiment, this method further comprising administering an anti-cancer agent. Representative agents which specifically block interaction between B7-H1 and CD80 but not interaction between B7-H1 and PD-1 include but are not limited to an antibody, a small inhibitor RNAi, an antisense RNA, a dominant negative protein, a small molecule inhibitor, or combinations thereof. A person having ordinary skill in this art would recognize that the antibody may be a monoclonal antibody or a functional fragment thereof, a humanized antibody or a functional fragment thereof, or an immunoglobulin fusion protein. In one preferred form, the antibody is the antibody designated 43H12. This method may be used to treat a variety of cancers and viral infections. Representative infections include but are not limited to infection with a hepatitis virus, a human immunodeficiency virus (HIV), a human T-lymphotrophic virus (HTLV), a herpes virus, an Epstein-Barr virus, or a human papilloma virus.

The present invention is still further directed to a composition comprising an agent which specifically blocks the interaction between B7-H1 and CD80 but does not block the interaction between B7-H1 and PD-1 and a vaccine, optionally in a pharmaceutically acceptable carrier. Represenative agents which specifically block interaction between B7-H1 and CD80 but not interaction between B7-H1 and PD-1 include but are not limited to an antibody, a small inhibitor RNAi, an antisense RNA, a dominant negative protein, a small molecule inhibitor, or combinations thereof. A person having ordinary skill in this art would recognize that the antibody may be a monoclonal antibody or a functional fragment thereof, a humanized antibody or a functional fragment thereof, or an immunoglobulin fusion protein. In one preferred embodiment, the antibody is the antibody designated 43H12.

The compositions of the present invention may be administered by any route desired, including but not limited to intravenously, or intramuscularly injecting to a subject the pharmaceutical composition in liquid form; subcutaneously implanting in said subject a pellet containing the pharmaceutical composition; or orally administering to the subject the pharmaceutical composition in a liquid or solid form.

The compositions of the present invention may be in the form of a pellet, a tablet, a capsule, a solution, a suspension, an emulsion, an elixir, a gel, a cream, a suppository or a parenteral formulation. The amount of the antibody administered would of course vary according to the size of the subject and various other factor but would typically be administered in a dose from about 0.01 mg/kg to about 100 mg/kg of the subject's body weight.

As used herein, the term “a” or “an”, when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, method steps, and/or methods of the invention. It is contemplated that any device or method described herein can be implemented with respect to any other device or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the terms “subject” or “individual” refers to any human or non-human recipient of the composition described herein.

The following example(s) are given for the purpose of illustrating various embodiments of the invention and are not meant to limit the present invention in any fashion.

### EXAMPLE 1

### Materials and Methods

### Mice

Female C57BL/6 (B6) and B6-background CD80-knockout (KO) mice were purchased from the National Cancer Institute (Frederick, Md.) and the Jackson Laboratory (Bar Harbor, Me.), respectively. OT-I TCR-transgenic mice were purchased from Taconic (Rockville, Md.). B6-background B7-H1-KO mice were generated by Dr. Lieping Chen (Johns Hopkins University). B7-H1-KO OT-I mice and CD8O-KO OT-I mice were generated by backcrossing OT-I transgenic mice with B7-H1-KO and CD8O-KO mice, respectively. The genotypes of these mice were validated by a flow cytometry using H-2K^{b}/OVA tetramer and PCR of genomic DNA. All mice were maintained under specific pathogen-free conditions and were used at 6-10 weeks of age.

### Peptide, Tetramer, and Antibodies

The OVA₂₅₇₋₂₆₄ peptide (SIINFEKL), an H-2K^{b}-restricted CTL epitope derived from chicken ovalbumin (OVA), was purchased from GenScript (Piscataway, N.J.). Anti-mouse B7-H1 mAb clone 43H12 was generated by immunizing Lewis rats with mouse B7-H1-Ig fusion protein according to an established method (22). Anti-mouse B7-H1 mAb clone 10B5 was established as described (23). Isotype-matched control rat IgG or hamster IgG were purchased from Rockland (Gilbertsville, Pa.). Allophycocyanin-conjugated anti-mouse CD8 mAb and FITC-conjugated anti-mouse CD80 mAb were purchased from eBioscience (San Diego, Calif.). PE-conjugated H-2K^{b}/OVA tetramer was purchased from Beckman Coulter (Fullerton, Calif.). FITC-conjugated anti-human IgG, anti-rat IgG, and anti-mouse IgG Abs were purchased from Invitrogen (Carlsbad, Calif.).

### Fusion Proteins

Recombinant proteins of mouse B7-H1 or PD-1 extracellular domain fused with human IgG1 Fc region were purchased from R&D (Minneapolis, Minn.). Chimeric genes of the extracellular domain of mouse CD80 or B7-DC (PD-L2, CD273) fused with mouse IgG2a Fc were constructed in pMIgV vector, as reported (24). Proteins were expressed in CHO cells by gene transfection and isolated by protein A affinity column. Similarly, fusion proteins of mouse B7-H3 or B7-H4 extracellular domains linked with human IgG1 Fc region were constructed in pHIgV vector, followed by expression and isolation (24). Purity of the isolated proteins was assessed by ELISA and SDS-PAGE. Biotin conjugation of fusion proteins was performed by using EZ-link Sulfo-NHS-Biotin reagents purchased from Thermo Scientific (Rockford, Ill.).

### Flow Cytometric Analysis and ELISA

Specific binding of 43H12 with mouse B7-H1 and its capability of selectively blocking B7-H1/CD80 interaction was assessed by flow cytometric analysis and ELISA, according to previous studies (25). In flow cytometry, Ab binding was detected by LSR II (BD Biosciences, San Jose, Calif.) and analyzed by FlowJo software (Tree Star, Inc. Ashland, Oreg.). In ELISA, Ab interaction with fusion proteins immobilized on ELISA plates was visualized by tetramethylbenzidine-based chromogenic assay and optical density (O.D.) at 450 nm was measured by Biotrak II plate reader (Amersham Biosciences, Cambridge, UK).

In vitro T Cell Proliferation Assay

In vitro T cell co-stimulatory assay was conducted as previously reported (26). Briefly, 96-well culture plates were first coated with 0.5 μg/ml anti-CD3 mAb and then with 10 μg/ml mouse B7-H1-human Fc fusion or control human Fc protein. Naïve T cells isolated from spleen and lymph nodes (LN) of CD80-KO mice were cultured in these wells at 1.5×10⁶ cells/ml in the presence of 10 μg/ml 43H12 or control rat IgG. Proliferative activity of T cells was assessed by an incorporation of ³H-thymidine during the last 6 hrs of 2 days culture.

### In Vivo Anergy Model of OVA-reactive OT-I Tcells

OT-I T cells were anergized by intravenous (i.v.) injection of OVA peptide, according to previous studies with some modifications (21,27). First, CD8⁺ T cells were isolated from the spleen and LN of wild-type (WT) OT-I, B7-H1-KO OT-I, or CD80-KO OT-I mice by negative selection using MACS (Miltenyi Biotech, Auburn, Calif.). Purity of the isolated OT-I CD8⁺ T cells was confirmed by a flow cytometry, and was constantly over 85%. The purified cells were injected i.v. into WT B6 mice or CD80-KO mice at a dose of 1×10⁶ cells/mouse. After 24 hrs, the recipient mice were injected i.v. with 0.5 mg OVA₂₅₇₋₂₆₄ peptide. For mAb treatment, mice were given intraperitoneally (i.p.) with 200 μg 43H12 or control rat IgG at the indicated time points. Spleen or PBMC were harvested later, and the percentage of OT-I CD8⁺ T cells was assessed by a flow cytometry.

Assays of in vivo BrdU Uptake and Annexin V Staining

CD8⁺ T cells from OT-I transgenic mice were transferred i.v. into B6 mice. After 24 hours, mice were given i.v. with 0.5 mg OVA₂₅₇₋₂₆₄ peptide and treated i.p. with 200 μg 43H12 or control rat IgG on day of peptide injection and 3 days later. Two or four days after peptide injection, the mice were treated i.p. with BrdU (100 μg/mouse, Sigma-Aldrich, St. Louis, Mo.), and the spleen was harvested 24 hrs after BrdU injection. BrdU incorporation OT-I CD8⁺ T cells was assessed by BrdU flow kit (BD Biosciences) along with staining by allophycocyanin-conjugated anti-CD8 mAb and PE-conjugated H-2K^{b}/OVA tetramer, according to the manufacturer's instructions.

For Annexin V staining, 1×10⁶ OT-I cells were transferred i.v. into B6 mice, which were then given i.v. with 0.5 mg OVA₂₅₇₋₂₆₄ peptide and treated i.p. with 200 μg 43H12 or control rat IgG, as described above. Four days after peptide administration, spleen was harvested and the percentage of Annexin V-positive cells in OT-I T cells was assessed by flow cytometry using FITC-conjugated Annexin V (BD Biosciences), allophycocyanin-conjugated anti-CD8 monoclonal antibody and PE-conjugated H-2K^{b}/OVA tetramer, according to the manufacturer's instructions.

### Induction and Aassessment of Oral Tolerance

B6 mice were given drinking water supplemented with 0.2 mg/ml OVA (Grade V, Sigma, St. Louis, Mo.) from day 0 to day 7. OVA-containing drinking water was replenished every other day. On day 14, the mice were immunized subcutaneously (s.c.) with 50 μg OVA emulsified in 50 μl CFA (Sigma). The mice were treated i.p. with 150 μg 43H12 or control rat IgG on day 0, 4, 8, and 12 for prevention model or on day 14 and 17 for recovery model. In both models, draining axillary and inguinal lymph nodes were harvested on day 21 and cultured in vitro at 2×10⁶ cells/ml in the presence of indicated doses of OVA protein (EndoGrade, Profos AG, Germany). The culture supernatants were harvested at indicated time points, and the concentrations of IFN-γ, IL-2, IL-4, and IL-17 were measured by ELISA kits (eBioscience). Proliferative activity of the incubated cells was assessed by ³H-thymidine incorporation during the last 10 hrs of 3 days culture.

### Statistical Analysis

Two-tailed student's t-test was used to compare two groups. P values <0.05 were considered significant.

### EXAMPLE 2

### Results

### Anti-B7-H1 mAb 43H12 Attenuates B7-H1/CD80 but not B7-H1/PD-1 Interaction

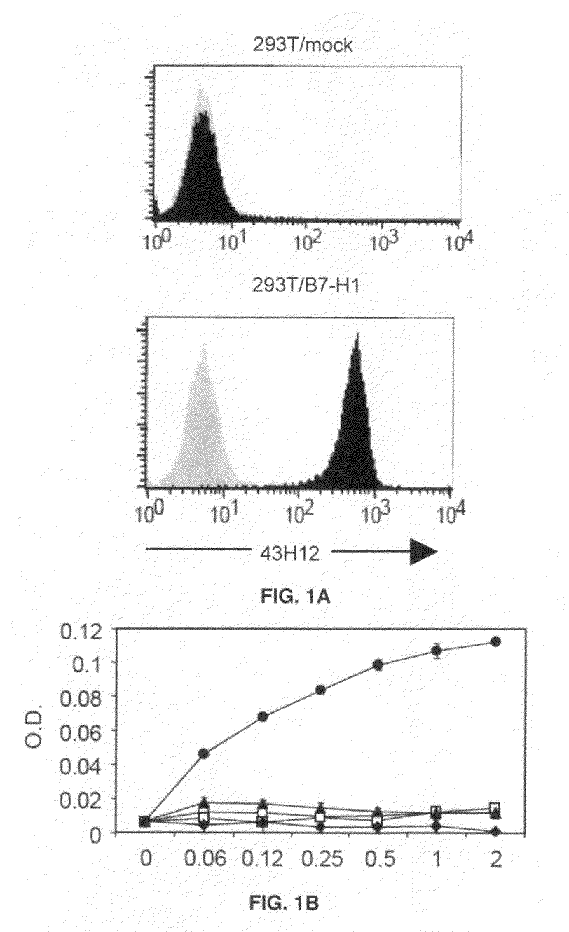

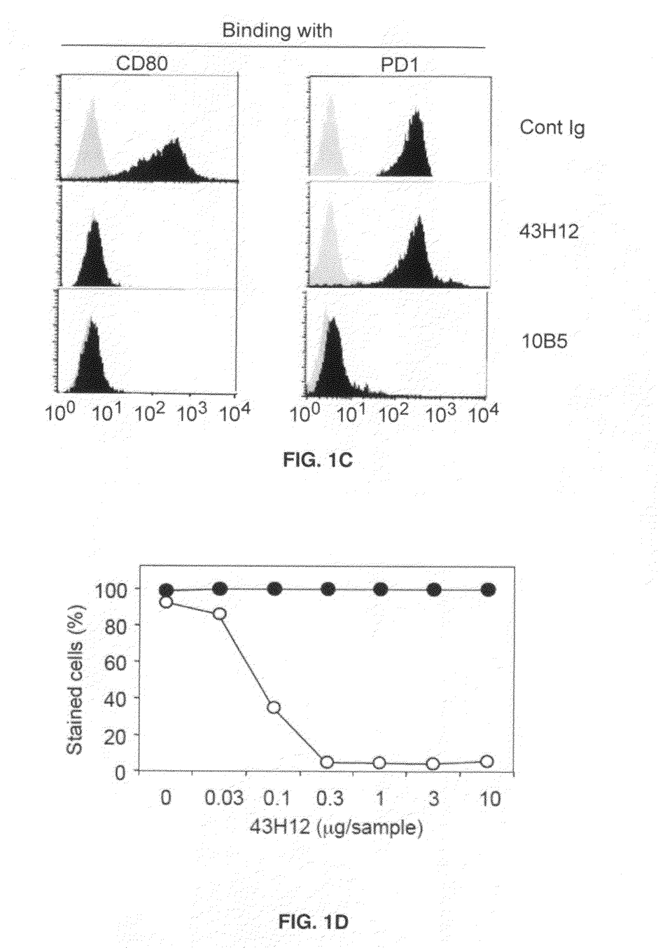

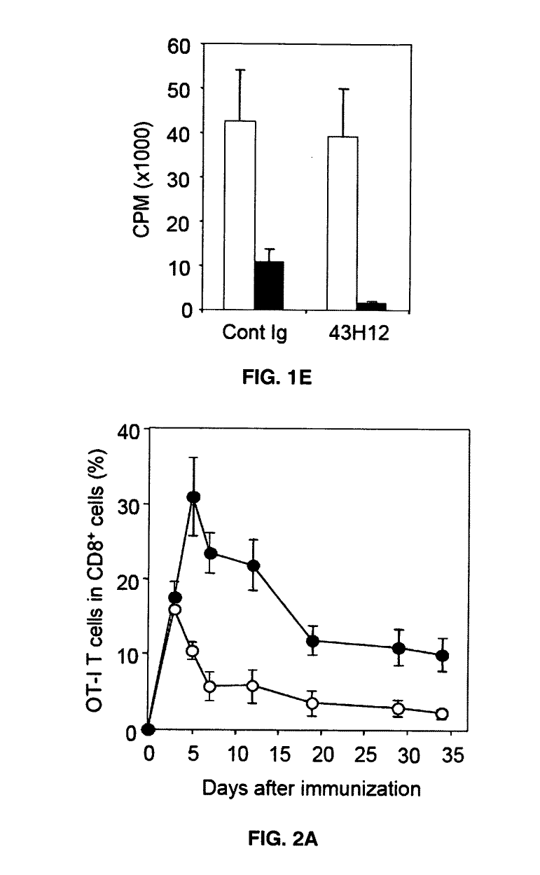

In order to elucidate immunological functions of the B7-H1/CD80 pathway in vivo, 43H12, a clone of anti-mouse B7-H1 monoclonal antibody which selectively interferes with B7-H1/CD80 but not B7-H1/PD-1 interaction was generated. Anti-mouse B7-H1 monoclonal antibody clone 43H12 was generated by immunizing Lewis rats with mouse B7-H1-Ig fusion protein emulsified with CFA or IFA every 2 weeks for total 3 times. Spleen cells from the immunized rats were harvested and fused with Sp2/0 myeloma cells so as to generate hybridoma cells. Clones were established by limiting dilution assay and those producing high level anti-B7-H1 monoclonal antibody were selected. Clone producing mAb that selectively interrupts B7-H1/CD80 but not 87-H1/PD-1 interaction was isolated and designated as 43H12. It has been known that binding sites of B7-H1 with PD-1 and CD80 are partially overlapped, but also contain the area which are selectively required for interaction with each molecule. The ability of 43H12 to selectively block B7-H1/CD80 but not B7-H1/PD-1 is probably associated with the characteristics that 43H12 binds and covers the area of B7-H1 surface required for interaction with CD80 but not PD-1. First, staining of 87-H1-expressing cells by 43H12 was confirmed by a flow cytometric assay (FIG. 1A). Specificity of 43H12 was assessed by ELISA, in which 43H12 showed a dose-dependent interaction with mouse B7-H1 protein but not with other B7 family proteins including CD80, B7-DC, B7-H3, and B7-H4 (FIG. 1B). Selective interaction of 43H12 with B7-H1 but not other B7 family molecules was also confirmed by a flow cytometry using cell lines expressing CD80 or CD86 (data not shown).

Next, the ability of 43H12 to block interactions between B7-H1 and its receptors was examined by flow cytometry. Inclusion of 43H12 completely abolished staining of mouse B7-H1-positive cells with mouse CD8O-Fc fusion protein, but not mouse PD-1-Fc protein (FIG. 1C). In contrast, other clones of anti-mouse B7-H1 monoclonal antibody including 10B5 and MIH5, which were previously developed (23,28) blocked both B7-H1/CD80 and B7-H1/PD-1 interactions (FIG. 1C and data not shown). The specificity of 43H12 to block B7-H1/CD80 was further tested by titration assay, in which as low as 0.3 μg 43H12 was sufficient to completely attenuate a binding of CD8O-Fc with B7-H1-expressing cells, while PD-1-Fc binding with the same cells was not interfered at all even with 10 μg 43H12(FIG. 1D).

In addition, the co-inhibitory effect of B7-H1-Fc protein on the proliferation of CD80-KO T cells was not abrogated in the presence of 43H12 (FIG. 1E), further indicating a negligible effect of 43H12 on the functions of B7-H1/PD-1 pathway. Thus, 43H12 is highly and selectively antagonistic to 67-H1/CD80 interaction, endorsing its capacity as a means to exploring B7-H1/CD80 functions. Interaction of 43H12 with B7-H1 did not modify expression levels of B7-H1 on cell surface, indicating that the effects of 43H12 are not caused by non-specific downregulation or internalization of B7-H1.

### Blockade of B7-H1/CD80 Interaction Enhances Aq-Specific T Cell Expansion

To explore in vivo functions of B7-H1/CD80 interaction, a model was employed in which OVA-reactive OT-I T cells undergo activation in response to i.v. injection of high-dose OVA₂₅₇₋₂₆₄ peptide. In this model, OT-I T cells show transient expansion followed by activation-induced apoptosis, i.e. contraction phase, and eventually undergo anergic status in chronic phase (27). Recent studies using this model revealed that treatment with 10B5, anti-B7-H1 blocking monoclonal antibody, accelerated expansion of OT-I T cell in early priming phase and resulted in prevention and recovery from T cell anergy (21). However, because 10B5 interferes with both B7-H1/PD-1 and B7-H1/CD80 interactions (FIG. 1C), a selective role of B7-H1/CD80 in T cell priming and anergy induction was unclear.

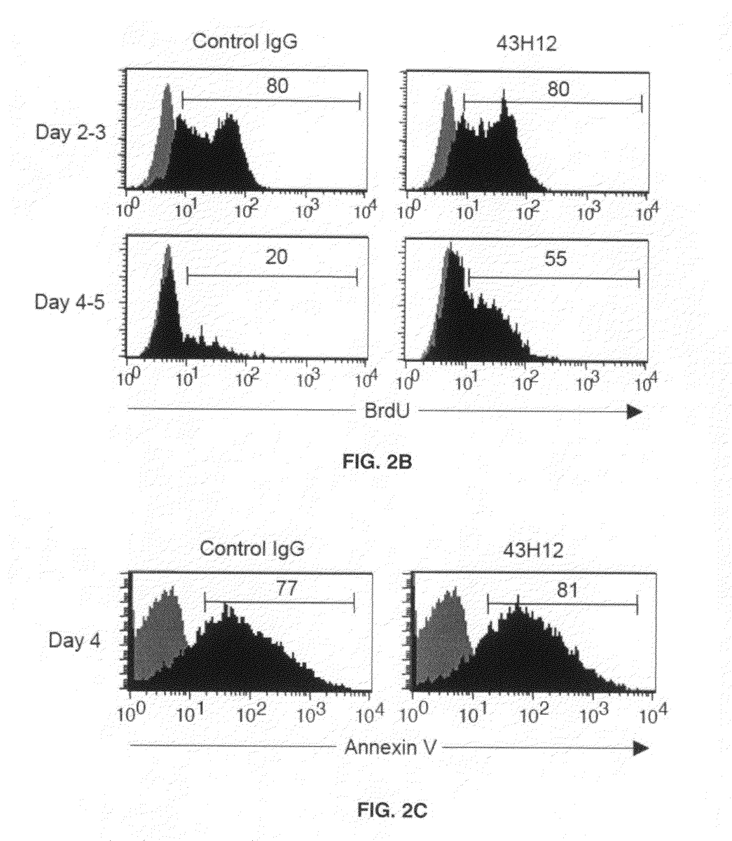

Therefore, this issue was examined by applying 43H12 to this model. 43H12 treatment significantly prolonged the expansion period of OT-I T cells, by which expansion peak shifted from day 3 to day 5 (FIG. 2A). 43H12 treatment induced OT-I T cell expansion up to 30% of total CD8⁺ T cells, which was twice that of control mice. Expansion level of OT-I T cells by 43H12 treatment was less drastic compared to the effect of 10B5 in the same model (21), demonstrating distinct features of these monoclonal antibodies, i.e., blockade of B7-H1/CD80 alone vs. dual blockade of B7-H1/CD80 and B7-H1/PD-1. In 43H12-treated mice, OT-I T cells gradually contracted after initial expansion, while the numbers of OT-I T cells were constantly higher than those in control Ig-treated mice (FIG. 2A). Thus, this result demonstrated an enhanced T cell response in the presence of 43H12, indicating an inhibitory function of B7-H1/CD80 interaction in T cell activation in vivo.

In order to explore the immunological mechanisms of the effects of 43H12, the in vivo proliferation and apoptosis of OT-I T cells was assessed by BrdU uptake and Annexin V staining assays. 43H12 treatment did not affect BrdU incorporation in OT-I T cells in the early expansion phase during day 2-3 after OVA injection (FIG. 2B). In control mice, OT-I T cells contracted its proliferation after day 3, and only 20% of them showed BrdU positive during day 4-5. In contrast, OT-I T cells in 43H12-treated mice sustained BrdU incorporation in which 55% of cells remained BrdU-positive in this time. These results were concordant with the finding that 43H12 treatment had no effects on OT-I T cell number until day 3, but it induced continuous expansion of OT-I T cells until day 5 after OVA injection (FIG. 2A). On the other hand, percentages of Annexin V-positive cells in OT-I T cells were comparable between 43H12- and control Ig-treated mice (FIG. 2C), suggesting a negligible role of 43H12 in T cell apoptosis. These results together indicate that signals delivered by B7-H1/CD80 interaction mediate inhibitory effects on late expansion phase of Ag-induced T cell responses.

### Effects of 43H12 are Mediated by Blockade of CD80 Signal in T Cells

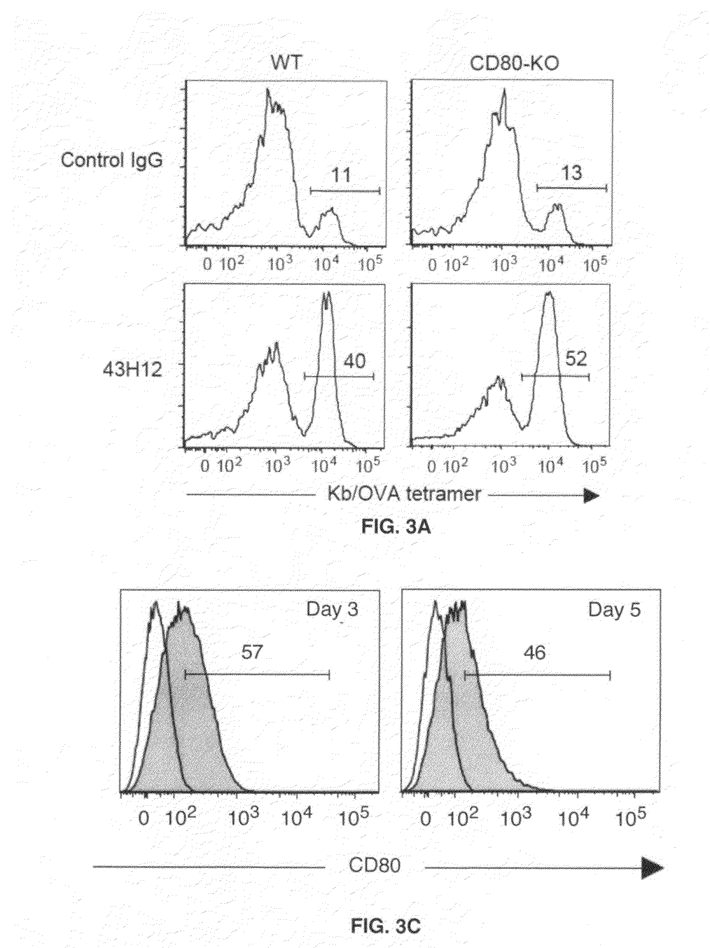

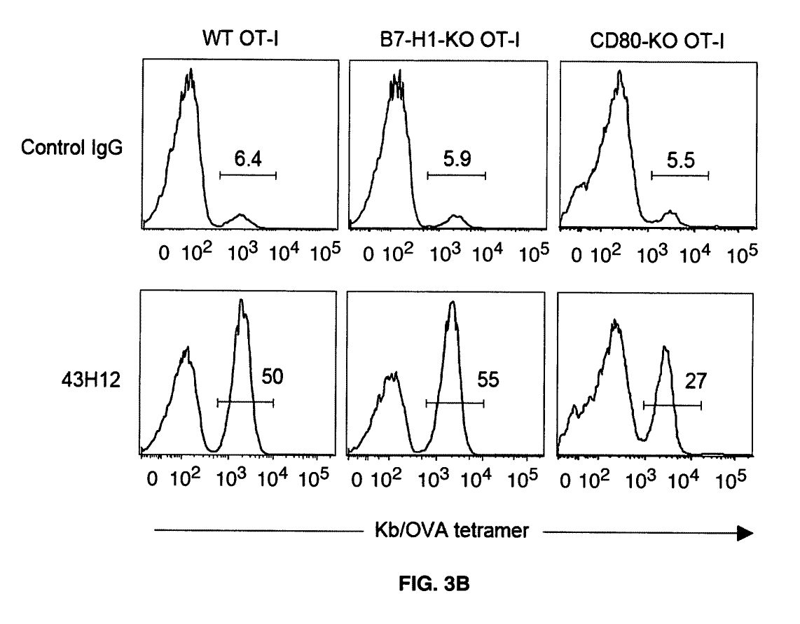

Previous studies indicated that B7-H1 and CD80 delivers inhibitory signals into T cells in bidirectional fashion (17). Therefore, which of B7-H1 or CD80, or both, are responsible as inhibitory receptor(s) on OT-I T cells was examined in this model. First, CD80-KO mice were employed as hosts of OT-I T cell transfer. In these conditions, expression of CD80 was ablated in all immune and non-immune cells other than donor OT-I T cells. 43H12 treatment induced profound expansion of OT-I T cells even in CD80-KO hosts at a level comparable to that observed in WT hosts (FIG. 3A). These results suggest that CD80 on non-T cells including antigen-presenting cells (APC) plays a dispensable role in the effects of 43H12. Since CD80-KO mice ablate its functions not only through B7-H1 but also CD28/CTLA-4 interactions, the effects of 43H12 were further tested in the presence or absence of anti-CD80 monoclonal antibody 16-10A1, a clone that blocks CD80 binding to CD28/CTLA-4 but not B7-H1 (18,29). OT-I T cell expansion induced by 43H12 treatment was not affected by an co-administration of 16-10A1. These results indicate that loss of CD80-CD28/CTLA-4 interaction does not manipulate the effects of 43H12, thus validating the findings in the model using CD80-KO mice. B7-H1-KO mice were used as hosts of OT-I T cell transfer in order to explore a role of B7-H1 on APC. However, OT-I T cells transferred into B7-H1-KO mice profoundly expanded without any treatments due to a loss of PD-1 signal as previously reported (21). Although 43H12 injection in such condition did not induce further expansion of OT-I T cells (data not shown), the high background number of OT-I T cells hindered assessment of a role of B7-H1 associated with APC. OT-I T cells deficient of B7-H1 or CD80 were transferred as donor cells into WT hosts. Treatment with 43H12 induced profound expansion of transferred B7-H1-KO OT-I T cells at a level comparable to WT OT-I donor T cells (FIG. 3B). On the other hand, expansion of CD80-KO OT-I T cells induced by 43H12 treatment was significantly lower than that of 43H12-treated WT OT-I T cells (27% vs. 50%), while it was still higher than control IgG treatment (27% vs. 5.5%). These results together suggest that the effects of B7-H1/CD80 blockade by 43H12 are dependent, at least in part, on CD80 expressed on donor OT-I T cells, but not B7-H1. To bolster this notion, CD80 expression on OT-I T cells was further assessed. Three and five days after OVA injection, CD80 was detected on approximately 50% of OT-I T cells (FIG. 3C), also supporting a potential role of CD80 in transmitting inhibitory signal to Ag-stimulated T cells.

### B7-H1/CD80 Interaction is Required for Induction and Maintenance of T Cell Anergy

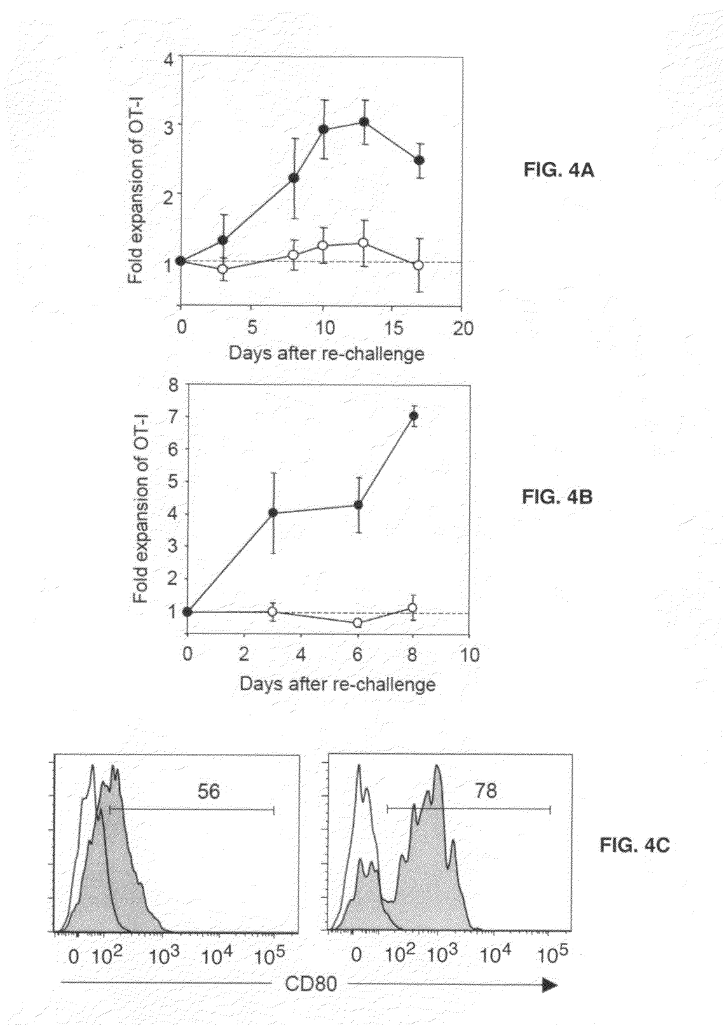

A regulatory role of B7-H1/CD80 interaction in T cell tolerance was next explored. It was previously reported that OT-I T cells undergo anergy following initial expansion and subsequent contraction in response to i.v. injection of OVA peptide in this model (21,27). Consistently, OT-I T cells in the host which was injected with OVA on day 0 and treated with control Ig on day 0 and 3 showed no detectable proliferation upon re-challenge of OVA on day 34 (FIG. 4A). In sharp contrast, OT-I T cells in the host which was treated with 43H12 on the day and three days after OVA injection expanded significantly in response to OVA rechallenge. These results indicate that blockade of B7-H1/CD80 interaction during T cell priming by tolerogenic Ag immunization prevents subsequent T cell anergy induction.

Next, whether blockade of B7-H1/CD80 interaction could also reverse pre-established T cell anergy was examined. Twenty days after initial OVA peptide injection, the mice harboring anergized OT-I T cells were re-challenged with OVA and simultaneously treated with either control Ig or 43H12 treatment. As expected, OT-I T cells in control Ig-treated mice did not show proliferative responses upon OVA re-challenge (FIG. 4B). In sharp contrast, 43H12 treatment together with OVA peptide re-challenge resulted in a profound expansion of OT-I T cells. These results suggest that blockade of B7-H1/CD80 interaction at the time of Ag re-encounter is capable of breaking pre-established T cell anergy.

In order to further support this conclusion, CD80 expression on anergic T cells was examined with or without Ag re-challenge. Twenty days after initial OVA injection, CD80 was expressed on approximately 50% of anergic OT-I T cells (FIG. 4C). This level of expression was comparable to those observed on day 3 and 5 after OVA injection (FIG. 3C), implicating that CD80 is continuously expressed on T cells after priming. When anergic OT-I T cells were exposed to OVA peptide re-challenge, CD80 expression was upregulated up to 80% within 24 hrs (FIG. 4C). Taken together, these results suggest that B7-H1/CD80 interaction plays a crucial role in induction and maintenance of anergic T cells, and that blockade of this interaction can prevent and reverse T cell anergy.

### A Crucial Role of B7-H1/CD80 Interaction in Induction and Maintenance of Oral Tolerance

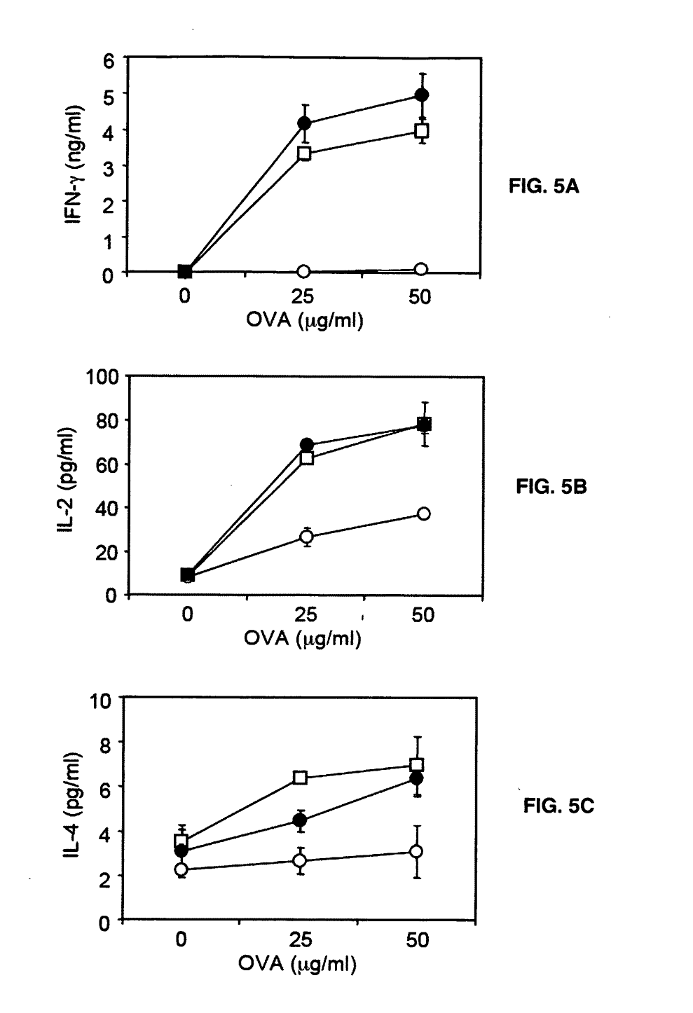

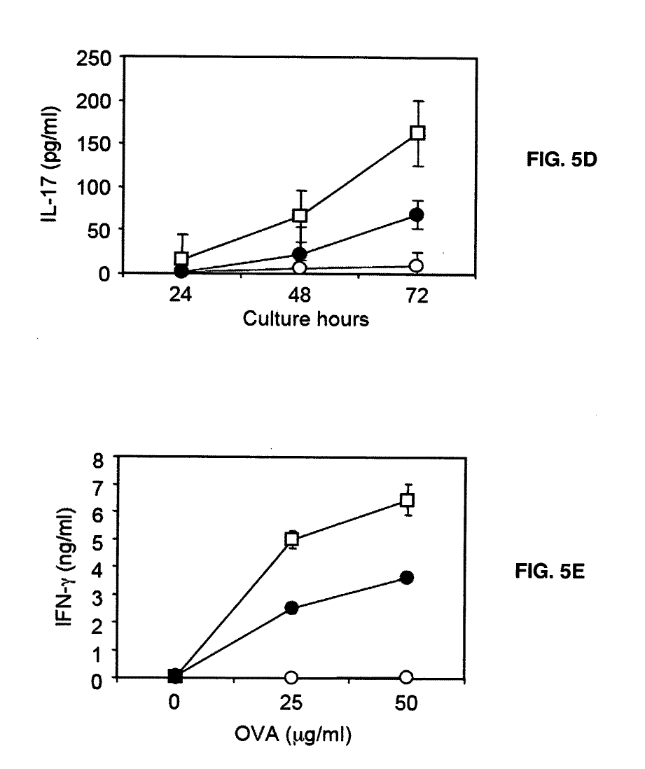

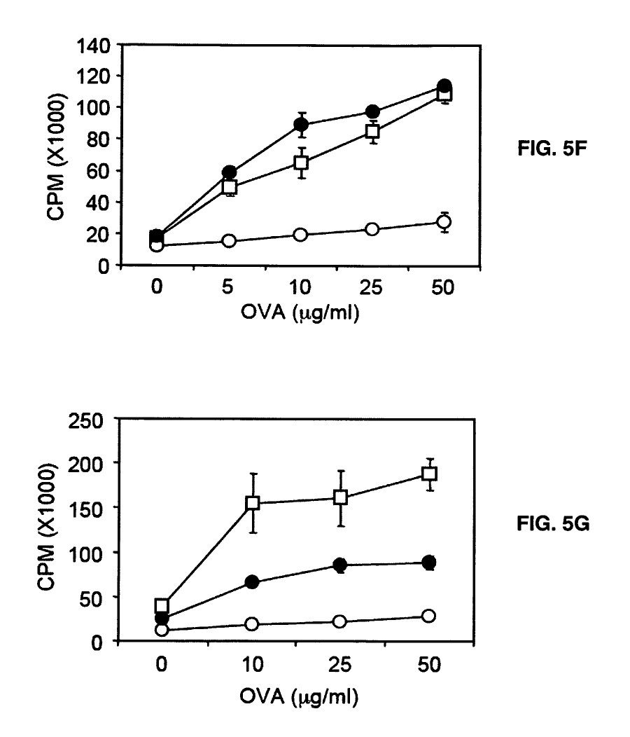

When Ag are given orally, the mucosal immune system in the gastrointestinal tract does not make productive responses but rather undergo Ag-specific tolerant condition, a process known as oral tolerance (30). This mechanism is essential for preventing deleterious immune reactions to self and exogenous dietary and environmental Ag such as food proteins. Although numbers of studies have investigated the mechanisms of oral tolerance, molecular checkpoints necessary for oral tolerance induction and maintenance are largely unknown. The present invention examined whether B7-H1/CD80 interaction has regulatory function in oral tolerance. As previously reported (31-32), oral administration of OVA protein significantly diminished T cell responses including proliferation and cytokine productions of IFN-g, IL-2, IL-4, and IL-17 which were induced by in vivo OVA/CFA immunization and subsequent in vitro re-stimulation with OVA protein (FIGS. 5A-5D, 5F). Treatment of mice with 43H12 during oral OVA administration restored OVA-reactive T cell proliferation (FIGS. 5F) and IFN-g/IL-2 productions almost completely to the level without oral tolerance. Production of IL-4 and IL-17 (FIGS. 5A-5B) was partially but significantly restored by 43H12 treatment (FIGS. 5C-5D). These results indicate that B7-H1/CD80 interaction is essential for the induction of oral tolerance. Treatment with 43H12 did not affect cellular compartment of intestinal intraepithelial lymphocytes (IEL), including CD8aa, TCRgd T cells, suggesting that the regulatory functions of B7-H1/CD80 pathway in oral tolerance are unlikely associated with its direct effects on gut-specific T cells.

Next, whether blockade of B7-H1/CD80 interaction could reverse T cell responses in the condition of pre-established oral tolerance was examined. The mice which had been given oral OVA administration were treated with 43H12 or control Ig at the time of OVA/CFA immunization. T cell proliferation (FIG. 5G) and IFN-g production (FIG. 5E) by in vitro OVA re-stimulation was partially but significantly restored by 43H12 injections. Taken together, these findings suggest that the B7-H1/CD80 interaction is a crucial regulator for the induction and maintenance of T cell tolerance induced by oral Ag administration, and blockade of this pathway results in prevention and reversal of oral tolerance.

Tumor-reactive CTL used are pmel (specific to B16 melanoma Ag gp100₂₅₋₃₃ presented on H-2K^{b}) and P1A (specific to P815 mastocytoma Ag P1A₃₅₋₄₃ presented on H-2L^{d}), which were obtained from the Jackson Laboratory and Dr. Yang Liu (University of Michigan), respectively are used to examine the functions of B7-H1/CD80 checkpoint in tumor-reactive CD8+ T cell responses. These CTL recognize bona fide tumor Ag which are endogenously expressed with a weak antigenicity. As to pmel mice, they have been crossed with B7-H1-KO or PD-1-KO mice (all of these mice are C57BU6 background), so as to generate B7-H1-KO pmel or CD80-KO pmel CTL. B7-H1-KO mice were generated as previously reported, while CD80-KO mice are purchased from the Jackson Laboratory.

### Functional Analysis of B7-H1/CD80 Pathway in Tumor-Reactive CTL Responses

The functions of B7-H1/CD80 pathway in tumor-reactive CTL are examined by employing in vitro culture systems. Isolated CD8+ T cells from pmel mice are cultured with irradiated spleen cells from syngeneic C57BU6 mice, in the presence of titrated doses of pmel Ag peptide gp100₂₅₋₃₃. In order to assess the role of B7-H1/CD80 pathway, the 43H12 mAb is included in the culture. After 2-5 days, activation and effector functions of pmel CTL are assessed by 1) proliferation (³H-thymidine incorporation), 2) cell cycle and death (by BrdU and 7-AAD staining kit), 3) cytokine production (by Cytometric Bead Array of Th1/Th2/Th17 kit to measure IL-2, IL-4, IL-6, IL-10, IL-17A, IFN-_, and TNF), 4) cytolytic activity (4 hr ⁵¹Cr-release assay using B16 melanoma as target cells). Analogous experiments using PIA CTL instead of pmel are performed, in which CTL which are cultured with irradiated syngeneic DBA/2 spleen cells in the presence of titrated doses of P1A₃₅₋₄₃ peptide. In this case, P815 mastocytoma expressing PIA Ag is used as target cells in the cytolytic assay. Thus, experimental groups are as follows: Group 1: pmel CTL/irradiated C57BU6 spleen cells/gp100 peptide/control Ab; Group 2: pmel CTL/irradiated C57BU6 spleen cells/gp100 peptide/43H12; Group 3: P1A CTUirradiated DBA/2 spleen cells/P1A peptide/control Ab; and Group 4: P1A CTUirradiated DBA/2 spleen cells/P1A peptide/43H12.

The suppressive functions of B7-H1/CD80 interaction are mediated by CD80 inhibitory receptor on OT-I CTL which interacts with B7-H1 ligand on APC. In the regulation of tumor-specific CTL, CD80 and B7-H1 also serves as a receptor and a ligand, respectively. In order to address this, cells from B7-H1-KO, CD80-KO, B7-H1-KO pmel, and CD80-KO pmel mice are used in the assays described above. That is, wild-type (VVT) pmel CTL are cultured with irradiated spleen cells from B7-H1-KO or CD80-KO mice and gp10025-33 peptide in the presence of 43H12 or control Ab. On the other hand, CD8+ T cells isolated from B7-H1-KO pmel or CD80-KO pmel mice are cultured with irradiated WT spleen cells and gp100₂₅₋₃₃ peptide in the presence of 43H12 or control Ab. Thus, experimental groups are as follows: Group 1: WT pmel CTUirradiated B7-H1-KO spleen cells/gp100 peptide/control antibody; Group 2: WT pmel CTUirradiated B7-H1-KO spleen cells/gp100 peptide/43H12; Group 3: WT pmel CTUirradiated CD80-KO spleen cells/gp100 peptide/control antibody; Group 4: WT pmel CTUirradiated CD80-KO spleen cells/gp100 peptide/43H12; Group 5: B7-H1-KO pmel CTUirradiated WT spleen cells/gp100 peptide/control antibody; Group 6: B7-H1-KO pmel CTUirradiated WT spleen cells/gp100 peptide/43H12; Group 7: CD80-KO pmel CTL/irradiated WT spleen cells/gp100 peptide/control antibody; and Group 8: CD80-KO pmel CTL/irradiated WT spleen cells/gp100 peptide/43H12.

B7-H1/CD80 co-signal pathway has inhibitory effects on tumor-reactive CTL. Thus, in the first set of experiments, inclusion of 43H12 enhances proliferation, cell cycle progression, cytokine production, and cytolytic activity of pmel and P1A CTL by attenuation of B7-H1/CD80 inhibitory co-signal. In the second set of experiments, 43H12 retains its effects in cases that CD80-KO spleen cells are used as APC (Group 3, 4) and B7-H1-KO pmel CTL are used as reacting cells (Group 5, 6), since B7-H1/CD80 checkpoint system remains intact in these combinations (it means, B7-H1 and CD80 serve as a ligand and a receptor, respectively). On the other hand, when B7-H1-KO spleen cells are used as APC (Group 1, 2) and CD8O-KO pmel CTL are used as reacting cells (Group 7, 8), the effect of 43H12 is abolished. These results will elucidate the inhibitory function of B7-H1/CD80 on tumor-reactive CTL and their roles as a ligand and a receptor.

Analysis of B7-H1/CD80 pathway in tumor-reactive CTL responses “in vivo”

The inhibitory effect of B7-H1/CD80 checkpoint pathway in tumor-reactive CTL is examined “in vivo”. To this end, pmel T cells are transferred intravenously (i.v.) into syngeneic C57BU6 mice which have been inoculated with B16 melanoma 7-10 days before (tumor size is 5-10 mm average when CTL are transferred). The mice are further treated with intraperitoneal (i.p.) injection of 43H12 or control antibody. Then, size of the tumor is measured periodically. In addition, the number of pmel CTL in tumor site and tumor-draining lymph nodes (LN), their expression of activation markers (CD44, CD25, CD62L, and CD69) and cytokines (Th1/Th2/Th17 Cytometric Bead Array) are analyzed. Population of pmel CTL in tumor site or tumor-draining LN are identified as Thy1.1-positive CD8-positive cells, since pmel mice from the Jackson Laboratory are Thy1.1-congenic (stock number: 005023). Similar experiments are performed with P1A CTL, in which DBA/2 mice bearing pre-established P815 tumor are injected with P1A CTL and treated with 43H12 or control Ab. To assess the number and functions, P1A CTL are identified as CD8, P1A/H-2L^{d} pentamer (Prolmmune)-double positive cells (22).

Next, the role of B7-H1 and CD80 as a ligand and a receptor in B7-H1/CD80 checkpoint function are addressed by in vivo experimental models. CTL isolated from B7-H1-KO pmel or CD8O-KO pmel are transferred i.v. into B7-H1-KO or CD80-KO host mice in which B16 melanoma is pre-established. The mice are injected i.p. with 43H12 or control antibody, and in vivo responses of pmel CTL and tumor growth is assessed as described above. Experimental groups will be composed as follows: Group 1: pmel CTL transferred into B7-H1-KO mice with B16 melanoma/treatment with control antibody; Group 2: pmel CTL transferred into B7-H1-KO mice with B16 melanoma/treatment with 43H12; Group 3: pmel CTL transferred into CD80-KO mice with B16 melanoma/treatment with control antibody; Group 4: pmel CTL transferred into CD80-KO mice with B16 melanoma/treatment with 43H12; Group 5: B7-H1-KO pmel CTL transferred into WT mice with B16 melanoma/treatment with control antibody; Group 6: 87-H1-KO pmel CTL transferred into WT mice with B16 melanoma/treatment with 43H12; Group 7: CD80-KO pmel CTL transferred into WT mice with B16 melanoma/treatment with control antibody; and Group 8: CD80-KO pmel CTL transferred into WT mice with B16 melanoma/treatment with 43H12.

The B7-H1/CD80 pathway has inhibitory effects on tumor-reactive CTL in vivo as well as in vitro. Thus, pmel and P1A CTL demonstrates increased cell number and enhanced expression of activation makers and cytokines after treatment with 43H12. 43H12 treatment retains its effects when CD80-KO mice are used as host mice (Group 3, 4) and when B7-H1-KO pmel CTL are transferred into VVT mice (Group 5, 6). In contrast, the effect of 43H12 on CTL responses are abolished when B7-H1-KO mice are used as host (Group 1, 2) and that CD8O-KO pmel CTL are transferred into WT mice (Group 7, 8).

The Role of B7-H1/CD80 Pathway in the Inhibition of Tumor-Reactive CD4+ T Effector Cells and their Conversion to iTreq Cells “in vivo”

The role of the B7-H1/CD80 co-signal pathway in TRP-1 CD4+ T cells “in vivo” was examined in terms of their activation, conversion to iTreg cells, and antitumor immune functions. First, B16 melanoma cells are inoculated subcutaneously (s.c.) into C57BU6 mice on day 0. After 7 days, the mice are exposed to 5 Gy-irradiation, and subsequently injected i.v. with naïve TRP-1 CD4+ T cells isolated from Thy1.1-positive TRP-1-specific TCR transgenic mice, as previously reported (33, 34). The mice are further treated i.p. with 43H12 or control Ab every 5 days. Thereafter, tumor size is measured periodically. In addition, the number of TRP-1 CD4+ T cells in tumor site and tumor-draining LN are assessed by detecting Thy1.1-positive CD4+ T cells. Expression of activation markers (CD44, CD25, CD62L, and CD69), and cytokines (intracellular staining of Th1/Th2/Th17-type cytokines) on TRP-1 CD4+ T cells are also examined. Finally, the number of TRP-1 T cell-derived iTreg cells are assessed as CD4/Thy1.1/Foxp3-triple positive population.

B7-H1/CD80 immune checkpoint system has inhibitory effects on tumor-reactive CD4+ T cells, it is expected that 43H12 treatment, which blocks B7-H1/CD80 pathway, will increase the number, activation status, and cytokine production of TRP-1 T cells. Accordingly, relapse of B16 melanoma, which is otherwise observed in 60% of mice is prevented due to an enhancement of antitumor activity of TRP-1 CD4+ T cells. In addition, 43H12 treatment reduces the emergence of Foxp3+ TPR-1 iTreg cells.

### Discussion

Recent studies revealed that B7-H1 binds CD80 besides PD-1, and the B7-H1/CD80 interaction delivers bidirectional co-inhibitory signals to T cells (17-18). However, a role of the B7-H1/CD80 interaction in T cell tolerance in physiological and pathological conditions remains unexplored. The present invention addresses this question by applying 43H12, a monoclonal antibody that selectively attenuates B7-H1/CD80 but not B7-H1/PD-1 interaction, to in vivo models of T cell activation and tolerance. Treatment with 43H12 enhanced T cell responses and consequently hindered induction and maintenance of T cell tolerance related to intravenous or oral administration of Ag. In this model, CD80, but not B7-H1, on Ag-reactive T cells is responsible at least in part for transmitting co-inhibitory signal. Thus, these findings revealed a regulatory mechanism of B7-H1/CD80 interaction in T cell immunity including peripheral tolerance.

Previous studies using chemical cross-linking analysis and molecular modeling approaches revealed that the binding site of B7-H1 with CD80 partially overlaps with that of PD-1 (17). In addition, binding affinity of B7-H1/CD80 (K_{D}˜1.7 mM) is weaker than that of B7-H1/PD-1 (K_{D}˜0.5 mM). These findings suggest that biological reagents or B7-H1 mutants which preferentially abrogate B7-H1/CD80 interaction while sparing B7-H1/PD-1 interaction are reasonable approaches to explore B7-H1/CD80 functions.

In the present invention, a novel clone of anti-B7-H1 monoclonal antibody, 43H12, was generated which blocks B7-H1/CD80 interaction with at least 30-fold higher specificity than B7-H1/PD-1 (FIG. 1D). In addition, binding of 43H12 does not induce internalization or downregulation of cell surface B7-H1. In functional levels, 43H12 does not interfere with T cell inhibition caused by B7-H1/PD-1 interaction (FIG. 1E), further supporting its credibility as a means to exploring selective functions of B7-H1/CD80 pathway.

According to the results of B7-H1-KO or CD80-KO mice used as hosts or the source of donor T cells, inhibitory signals mediated by B7-H1/CD80 interaction are dependent in part on CD80 expressed on Ag-reactive T cells but not on non-T cells such as APC (FIG. 3A-3B). Consistently, CD80 expression on the primed and anergic T cells was detected in these models (FIG. 3C and FIG. 4C). A role of CD80 on T cells as an inhibitory receptor to deliver outside-in signal is concordant with previous findings including 1) increased cytokine productions in CD80-KO T cells, 2) an enhanced severity of graft-versus-host disease by CD80/CD86-KO donor T cells, and 3) resistance of CD80-KO T cells to inhibitory effects of T regulatory cells (Treg) (33-35). In addition, a cross-link of CD80 by anti-CD80 mAb induces growth retardation and upregulated expressions of pro-apoptotic molecules in lymphoma (36), providing more direct evidence of inhibitory signal transduction through CD80. Interestingly, 43H12 treatment induced a partial stimulation even in CD80-KO OT-I T cells (FIG. 3B), implicating a possibility of currently unknown non-CD80/non-PD-1 inhibitory receptor(s) of which interaction with B7-H1 is susceptible to blockade by 43H12.

In contrast to CD80, B7-H1 on Ag-reactive T cells plays a negligible role in these models (FIG. 3B). Possible cellular sources of B7-H1 on non-T cells include APC, Treg, myeloid-derived suppressor cells, and non-hematopoietic parenchymal cells. B7-H1 is ubiquitously expressed on these types of cells and recognized to induce immune tolerance via direct inhibition of T cells or generation of adaptive/induced Treg, while it has yet to be fully explored whether PD-1, CD80, or both receptors play a responsible role in these effects (6,37-41). In addition, B7-H1 expressed on non-T cells may also deliver outside-in signal as previously reported (42-43). Taken together, a role of B7-H1/CD80 signals in T cell tolerance is likely dependent on both T cell intrinsic and extrinsic mechanisms. Although it is currently unclear why T cell-associated B7-H1 is dispensable in spite of its capability of delivering T cell inhibitory signal by CD80 ligation (17), this discrepancy is probably due to some crucial differences in experimental systems (in vitro vs. in vivo) and target cells (CD4⁺ vs. CD8⁺ T cells) between these studies.

Presentation of high-dose Ag without adjuvants or tolerogenic APC leads to transient expansion of Ag-specific T cells and subsequent contraction, followed by generation of long-term T cell anergy. Negative co-signaling molecules including CTLA-4 and PD-1 play a crucial role in these processes of T cell tolerance (21,37). The present invention teaches that B7-H1/CD80 interaction also contributes to T cell tolerance generation, although its physiological role and mechanism are distinct from that of B7-H1/PD-1 interaction. First, as previously reported, B7-H1/PD-1 signaling showed regulatory effects on early phase (˜48 hrs) T cell responses after Ag encounter (20-21). In contrast, the present invention disclosed that blockade of B7-H1/CD80 interaction has negligible effects on T cell responses until 3 days after Ag stimulation, but rather continuously stimulates T cell expansion after 3 days (FIGS. 2A-2B). Thus, B7-H1/CD80 signal has inhibitory effects on the late stage of T cell responses which could regulate phase transition from T cell expansion to contraction. Second, CD80 expression is maintained on anergic T cells for relatively long period and quickly upregulated by Ag re-exposure to the level higher than that on primed T cells (FIGS. 3C and FIG. 4C). Furthermore, B7-H1/CD80 interaction is prerequisite for maintenance of anergic phenotype of T cells (FIG. 4B). Thus, CD80 expression may serve as a biomarker and functional checkpoint for T cell anergy, while similar features have been suggested with lymphocyte activation gene-3 (LAG-3) and BTLA (44-45). On the other hand, B7-H1/PD-1 interaction plays a crucial role in the induction and maintenance of T cell exhaustion (19).

Oral tolerance is the physiologic mechanism by which the mucosal immune system prevents adverse T cell responses against self and exogenous dietary Ag (30). Among co-signal pathways, CD80/CD86-CTLA-4 and B7-DC (PD-L2)-PD-1 have been shown to contribute to oral tolerance regulation (31,46-49). The present invention demonstrated that B7-H1/CD80 interaction also plays a crucial role in the induction and maintenance of oral tolerance (FIGS. 5A-5G). This notion could be supported by recent reports that B7-H1 is highly expressed on CD11c⁺ CD8a⁻ DC in mesenteric LN, which are a vital mediator for oral tolerance (31,50-51). While various mechanisms have been reported in oral tolerance of Ag-reactive CD4⁺ T cells, one of primary determinants is quantity of orally administered Ag. High doses of Ag induce T cell anergy, while low doses of Ag favor suppression-type tolerance caused by Treg or suppressor T cells which produce inhibitory cytokines such as TGF-b, IL-10, and IL-4 (30). Since the dose used in the present invention falls within low dose range, B7-H1/CD80 interaction may regulate suppression mechanisms of CD4⁺ T cells.

Blockade of B7-H1 functions is expected to have significant clinical value as a novel immunotherapy for diseases including cancer and chronic infection. The current studies give an insight into the complexity of these approaches which could affect B7-H1/PD-1, B7-H1/CD80, or both of them according to the reagents to be employed. For example, while selective attenuation of B7-H1/CD80 may have weaker effects compared to non-selective B7-H1 blockade, it could be advantageous in terms of minimizing a risk of autoimmune responses. In addition, the present invention indicates that blockade of the B7-H1/CD80 inhibitory signal could be utilized as an adjuvant for oral vaccine. In summary, the present invention revealed a crucial role of the B7-H1/CD80 pathway in the induction and maintenance of T cell tolerance and propose a therapeutic potential of blocking this pathway for prevention and restoration of peripheral T cell tolerance.

The following references may have been cited herein:
1. Chen L. Nat Rev Immunol. 2004;4:336-347.
2. Dong et al., Immunity. 2004;20:327-336.
3. Latchman et al., Proc Natl Acad Sci U S A. 2004;101:10691-10696.
4. Zhu et al. J Immunol. 2006;176:3480-3489.
5. Reynoso et al. J Immunol. 2009;182:2102-2112.
6. Keir et al. J Exp Med. 2006;203:883-895.
7. Brown et al. J Immunol. 2003;170:1257-1266.
8. Schreiner et al., Eur J Immunol. 2008;38:2706-2717.
9. Okazaki et al., Proc Natl Acad Sci U S A. 2001;98:13866-13871.
10. Chemnitz et al., J Immunol. 2004;173:945-954.
11. Parry et al. Mol Cell Biol. 2005;25:9543-9553.
12. Prokunina et al. Nat Genet. 2002;32:666-669.
13. Nielsen et al., Tissue Antigens. 2003;62:492-497.
14. Kong et al. Arthritis Rheum. 2005;52:1058-1062.
15. Newby et al. 2007;67:125-128.
16. Okazaki T, Honjo T. Trends Immunol. 2006;27:195-201.
17. Butte et al., Immunity. 2007;27:111-122.
18. Butte et al., Mol Immunol. 2008;45:3567-3572.
19. Barber et al., Nature. 2006;439:682-687.
20. Goldberg et al., Blood. 2007;110:186-192.
21. Tsushima F, Yao S, Shin T, et al. Blood. 2007;110:180-185.
22. Wilcox, et al., J Clin Invest. 2002;109:651-659.
23. Hirano et al. Cancer Res. 2005;65:1089-1096.
24. Chapoval Al, et al., Mol Biotechnol. 2002;21:259-264.
25. Anand et al. J Clin Invest. 2006;116:1045-1051.
26. Tamada et al. Nat Med. 2000;6:283-289.
27. Wilcox et al., Blood. 2004;103:177-184.
28. Aramaki 0, Shirasugi N, Takayama T, et al. Transplantation. 2004;77:6-12.
29. Razi-Wolf et al., Proc Natl Acad Sci USA. 1992;89:4210-4214.
30. Faria A M, Weiner H L. Oral tolerance. Immunol Rev. 2005;206:232-259.
31. Zhang Y, et al. Proc Natl Acad Sci U S A. 2006;103:11695-11700.
32. Arnaboldi et al., Mucosal Immunol. 2009;2:427-438.
33. Schweitzer A N, J Immunol. 1999;163:4819-4825.
34. Taylor et al., J Immunol. 2004;172:34-39.
35. Paust et al., Proc Natl Acad Sci U S A. 2004;101:10398-10403.
36. Suvas et al., J Biol Chem. 2002;277:7766-7775.
37. Probst et al., Nat Immunol. 2005;6:280-286.
38. Kitazawa et al. Transplantation. 2007;83:774-782.
39. Dong et al., Nat Med. 2002;8:793-800.
40. Wang et al., Proc Natl Acad Sci U S A. 2008;105:9331-9336.
41. Francisco et al. J Exp Med. 2009.
42. Azuma et al., Blood. 2008;111:3635-3643.
43. Dong et al., J Clin Invest. 2003;111:363-370.
44. Huang C T, Workman C J, Flies D, et al Immunity. 2004;21:503-513.
45. Hurchla et al. J Immunol. 2005;174:3377-3385.
46. Desvignes et al., J Immunol. 2000;164:2515-2522.
47. Zheng et al., J Immunol. 2004;172:2778-2784.
48. Samoilova et al., Int Eur J Immunol. 2002;32:2997-3006.
50. Chung et al., Blood. 2005;106:201-206.
51. Goubier A, Dubois B, Gheit H, et al. Immunity. 2008;29:464-475.

Any patents or publications mentioned in this specification are indicative of the levels of those skilled in the art to which the invention pertains. These patents and publications are incorporated by reference herein to the same extent as if each individual publication was incorporated by reference specifically and individually.

One skilled in the art will appreciate that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

What is claimed is:

1. An anti-B7-H1 monoclonal antibody which specifically blocks interaction between B7-H1 and CD80 but does not block interaction between B7-H1 and PD-1.

2. The anti-B7-H1 monoclonal antibody of claim 1, wherein said antibody blocks B7-H1/CD80 interaction with at least 30-fold higher specificity than B7-H1/PD-1.

3. The anti-B7-H1 monoclonal antibody of claim 1, wherein said antibody is 43H12.

4. A method of enhancing T cell expansion and decreasing T cell anergy induction in an individual in need of such treatment, comprising the step of:
administering to said individual an effective amount of a monoclonal antibody of claim 1 which specifically blocks interaction between B7-H1 and CD80 but does not block interaction between B7-H1 and PD-1.

5. The method of claim 4, wherein administration of said antibody results an enhanced T cell response.

6. The method of claim 4, wherein administration of said antibody decreases an inhibitory effect on late expansion phase of Ag-induced T cell responses and decreases T cell anergy induction.

7. The method of claim 4, wherein administration of said antibody increases production of IL-4 and IL-17.

8. A method of enhancing efficacy of a vaccine comprising administering an agent which specifically blocks interaction between B7-H1 and CD80 but does not block interaction between B7-H1 and PD-1.

9. A method of treating or inhibiting abnormal cell proliferation or a viral infection in a host comprising the step of:
administering an agent which specifically blocks interaction between B7-H1 and CD80 but does not block interaction between B7-H1 and PD-1 in combination with a vaccine against the cancer to a host in need thereof.

10. The method of claim 9, further comprising administering an anti-cancer agent.

11. The method of claim 9 wherein the agent is an antibody, a small inhibitor RNAi, an antisense RNA, a dominant negative protein, a small molecule inhibitor, or combinations thereof.

12. The method of claim 11, wherein the antibody is a monoclonal antibody or a functional fragment thereof, a humanized antibody or a functional fragment thereof, or an immunoglobulin fusion protein.

13. The method of claim 12, wherein said antibody is 43H12.

14. The method of claim 9, wherein the viral infection is a an infection with a hepatitis virus, a human immunodeficiency virus (HIV), a human T-lymphotrophic virus (HTLV), a herpes virus, an Epstein-Barr virus, or a human papilloma virus.

15. A composition comprising an agent which specifically blocks interaction between B7-H1 and CD80 but does not block interaction between B7-H1 and PD-1 and a vaccine, optionally in a pharmaceutically acceptable carrier.

16. The composition of claim 15 wherein the agent is an antibody, a small inhibitor RNAi, an antisense RNA, a dominant negative protein, a small molecule inhibitor.

17. The method of claim 16, wherein the antibody is a monoclonal antibody or a functional fragment thereof, a humanized antibody or a functional fragment thereof, or an immunoglobulin fusion protein.

18. The method of claim 17, wherein said antibody is 43H12.
